(19) United States
(12) Patent Application Publication
(10) Pub. No.: US 2018/0224421 A1
(43) Pub. Date: Aug. 9, 2018

(54) METHOD AND APPARATUS FOR A SURFACE MICROSTRUCTURED MULTIELECTRODE ARRAY

(71) Applicant: Robert John Petcavich, The Woodlands, TX (US)

(72) Inventor: Robert John Petcavich, The Woodlands, TX (US)

(21) Appl. No.: 15/887,441

(22) Filed: Feb. 2, 2018

Related U.S. Application Data
(60) Provisional application No. 62/454,473, filed on Feb. 3, 2017.

(51) Int. Cl. G01N 33/483; B01L 3/00; G01N 33/50

(52) U.S. Cl. CPC G01N 33/4836; B01L 3/5085; B01L 2300/0645; B01L 2300/0829; G01N 33/502

(57) ABSTRACT

The present patent application discloses, among other things, methods and apparatus for a surface microstructured multielectrode array (MEA) for enabling the auto alignment of living cells on exposed electrodes. In one approach the microstructures are fabricated onto the surface of a multielectrode array and attached to a multi well plate such that the metal electrodes are exposed for the attachment of cells to the surface. The microstructure allows for the living cells to self organize into biologically relevant organ mimics onto the electrodes only, which allows electronic electrophysiology monitoting of their behavior when exposed to, e.g., therapeutic substances.

### CROSS-REFERENCE TO RELATED APPLICATIONS

This application claims the benefit of the filing date of U.S. application Ser. No. 62/454,473, filed on Feb. 3, 2017, the disclosure of which is incorporated by reference herein.

### SUMMARY

A microelectrode array (MEA) of the present assay apparatus is a grid of tightly spaced electrodes in a planar array at the bottom of a cell culture plate. Electrically active cells, such as neurons or cardiomyocytes, can be cultured over the electrodes. Over time, as the cultures become established, they form cohesive networks and present an electrophysiological profile. The resulting electrical activity spontaneous or induced firing of neurons, or the uniform beat of cardiomyocytes is captured from each electrode on a microsecond timescale providing both temporally and spatially precise data. In contrast to traditional electrophysiology approaches such as patch clamp, the electrical activity measured on each MEA electrode is the total extracellular change in ions, reported as changes in voltage. “Extracellular field potential” provides access to electrophysiological data without disrupting the cellular membrane (non-invasive) or requiring dyes (label-free). Thus, MEA recordings can be taken over time on the same culture (hours-long continual data collection or repeated reads of the same plate over days, hours, or months), an advantage traditional techniques cannot provide.

Additionally, each electrode on the microelectrode array is capable of recording or stimulating the overlying cell culture allowing the monitoring and control of cellular network behavior in each well. These capabilities enable better exploration of complex biological networks, and learning about how a cell's circuitry functions together further advancing applications such as disease modeling, stem cell development, drug discovery and safety/toxicity testing. MEA systems that can be used in the present approach include, but are not limited to, those obtained from Axion Biosystems (Atlanta Georgia), ACEA Biosystems Inc. (San Diego, Calif.), and. Applied Biophysics Inc. (Troy N.Y.), to name just a few.

One advantage of certain embodiments of the present subject matter is that a self-organized biologically relevant in vitro cell model enabled by a microstructured surface fabricated onto a multi electrode array circuit board is provided.

Another advantage of certain embodiments of the present subject matter is that they enable fabrication of a microstructured surface on a multi electrode array whereby the tnicrostructured pattern is deposited on all regions of the MEA circuit board except the electrodes.

Another advantage of certain embodiments of the present subject matter is a microstructured surface on a multi electrode array whereby the microstructured pattern has dimensions that allow for the spaces over the electrode patterns to be at least on the order of a cell diameter.

Yet another advantage of certain embodiments of the present subject matter is a microstructured surface on a MEA whereby the micro pattern height contains the cells into the spaces over the unpatterned electrodes.

Another advantage of certain embodiments of the present subject matter is a microstructured surface on a MEA circuit board fabricated by photolithography or micro embossing.

Yet another advantage of certain embodiments of the present subject matter is a microstructured surface patterned MEA circuit board that subsequently is attached to, e.g., affixed to using an adhesive, a microwell array plate. For example, the microwell plate is disposed on a microstructured surface patterned MEA. In one embodiment, the microwells, which do not have a bottom so that cells placed in the wells can be in contact with the electrodes, include the microstructures. in some embodiment, the electrodes may be coated with a material that enhances cell binding, e.g., extracellular matrix.

In one embodiment, the disclosure provides a surface micro structured multi electrode array that is integrated into a microwell plate for measuring electrophysiological induced changes on self-aligned mammalian cells that have been exposed to therapeutic compounds.

Other advantages of the present subject matter will be apparent to a person of skill in the art upon reading and understanding this patent application.

### BRIEF DESCRIPTION OF THE DRAWINGS

FIG. 1 shows a top view of the surface of a multi electrode array that is fabricated and subsequently attached to the bottom of a microwell plate, according to one embodiment of the present subject matter.

FIG. 2 shows a top view of a multi electrode array onto which a micro pattern has been fabricated leaving the metal electrodes exposed for subsequent cell attachment, according to one embodiment of the present subject matter.

### DETAILED DESCRIPTION

The following discussion is directed towards the various embodiments of the present subject matter. Although one or more of these embodiments may be preferred, the embodiments disclosed should not be interpreted, or otherwise used, as a limiting the scope of the disclosure, including the claims. In addition one skilled in the art will understand that the following description has broad application, and the discussion of any embodiment is meant only to be exemplary of that embodiment, and not intended to intimate that the scope of the disclosure, including the claims, is limited to that embodiment.

Commercially available products and other prior art fail to teach how to micro pattern the surface of an MEA electrode circuit board for cell containment or auto alignment in order to induce more biologically relevant organ mimics on the surfaces of the MEA for electronic analysis.

Some embodiments of the present subject matter are directed to using multi electrode arrays (MEAs) in combination with microstructured topological surface features that are layered on top of the MEAs to align living cells that are attached to the surface. The structured MEA electrodes measure electrophysiological changes in aligned or patterned cells such as cardiomyocytes in contact with the MEA electrodes. It has been discovered that micro alignment of cardiac cells leads to maturing the cells into a more biologically relevant model of human heart tissue. As a result of this surface microstructured MEA, the cells self-align on the MEA electrodes, and electrophysiological changes can be measured in real time when the MEA array is exposed to various therapeutic compounds.

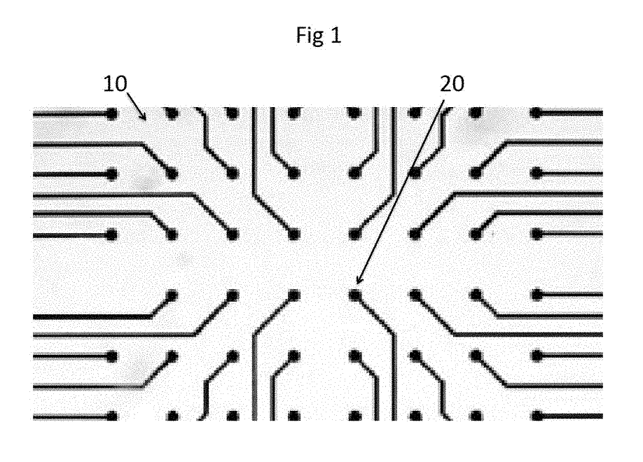

FIG. 1 shows a top view of one embodiment of the present subject matter. This exemplary embodiment is comprised of a dielectric substrate or support 10 onto which micro metal electrodes 20 have been patterned. In certain embodiments, the MEA electrodes are arranged and disposed onto an area of a microwell plate. In some embodiments, the electrodes are arranged and confined to a predetermined area of the microwell plate. In some embodiments the predetermined area of the tnicrowell plate is a known and commonly employed area. The microwell plate can have n×n wells where n=1-100. The number of electrodes in each well can vary from, for example, n=1 to n=100 with n=(2-16) being one embodiment. Other ranges of numbers of electrodes may be used without departing from the scope of the present subject matter, such as n=(2-32), n=(2-64), n=(16-64), and n=(64-100).

The electrode support 10 can consist of a glass, silicon, standard printed circuit board (PCB), or flexible polymeric film such as Kapton, polycarbonate, or polyester (PET) film. The thickness of the support 10 can range from 1 micron to 2 millimeters with 25 to 250 microns being one embodiment. The support 10 can be opaque or transparent and in one embodiment a transparent support is utilized. In certain embodiments, the transparent support is PET. Other materials and opacities may be used.

The conductive electrodes 20 are comprised of a conductor such as copper, silver, gold, nickel, aluminum, platinum, palladium, indium tin oxide, graphene, carbon na.notubes, carbon nanobuds, and silver nanowires. The electrodes 20 should have an electrical resistivity of less than 100 ohms per square with less than 10 ohms per square being one embodiment, The electrodes can be patterned in any geometric shape or size width lines, and interdigitated conductive lines being one embodiment. The width of the lines can vary from 1 to 300 microns with 50 to 100 microns being one embodiment. In one embodiment gold coated copper electrodes 10 that have been flash plated with gold to make the surface more biologically compatible for cell attachment and viability are employed.

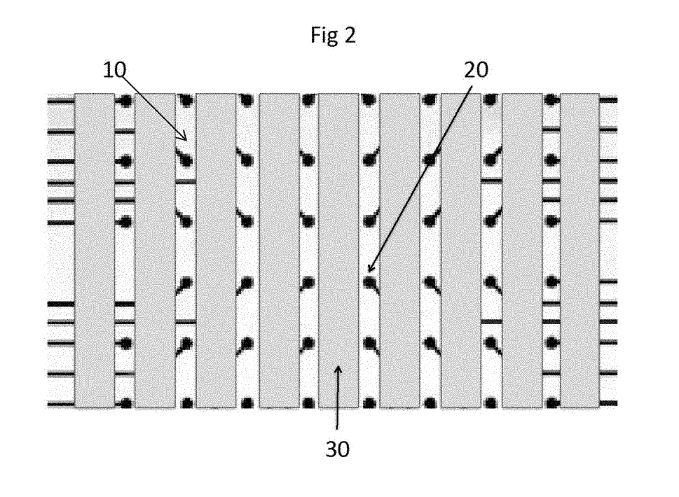

In various embodiments, the multielectrode array 20 is fabricated on a support material 10, and then a dielectric micro pattern 30 (see, e.g., FIG. 2) is fabricated onto the MEA electrodes such that the micro pattern 30 does not cover the metal electrodes. In certain embodiments, the multielectrode array 20 is fabricated by techniques known in the art.

In various embodiments of the present subject matter, it was found that a micro pattern 30 alines with dimensions that range from 10-60 microns in width and 5-50 microns in height with channels or spaces between the lines on the order of 20-70 microns was particularly usefut In certain embodiments, the micro pattern 30 is fabricated by photolithographic techniques known in the art using standard photoresist technology such as SUS available from the MicroChem, Newton, Mass. USA. However, the patterns can also be fabricated using micro embossing and micro Flexographic printing, both of which are known in the art. The micro pattern can have a smooth surface or be comprised of or have a polygon texture to the surface such as hexagons, pentagons or circular features.

In various embodiments, the next step is to attach the cells of interest to the exposed MEA electrodes. Good cell adhesion and attachment are critical for a cell functioning, viability and measurement of the electrophysiology of the neurons during therapeutic drug exposures of the stack. In various embodiments, gold-coated copper electrodes 20 are plasma cleaned to remove any surface contamination and then reacted with a 20 mM solution of alkanethiols of 11-mercaptoundecanoic acid (MUA) for 5 to 10 minutes. This results in a self assembled monolayer (SAM) or MUA on the surface. The electrodes are then immersed into a 150 mM solution of 1-ethyl-3-(3-dimethylamino-propyl) carbodiimide (EDAC) and 30 mM N-hydroxysuccinimide (NHS) for 30 minutes to attach the NHS group to the terminus —COOH of the SAM layer, The finished activated electrode structure is then sterilized with 70% ethanol for 15 minutes and exposed to various proteins that have well known binding sites for cells. For example the protein or polypeptides used can be fibronectin, laminin, Arg-Glu-Asp-Val-Tyr (REDV) or Lys-Arg-Glu-Asp-Val-Try (KREDVY) are just a few examples of polypeptide cell binding molecules. In one embodiment KREDVY is employed to enhance cell binding and viability after cell attachment. In the exemplary embodiment cardiomyocytes are attached to the electrodes using the aforementioned protocols with the addition of Matrigel to the surface to enhance cell attachment and viability. This allows the cells to mature and age during a drug-screening assay to observe the effect of cell maturing on drug response. Other cell types can also be used such as neuron progenitors, neurons, fibroblasts, endothelial cells, epithelial cells, liver, kidney, pancreas or dorsal root ganglion cells. Using the aforementioned protocols it is possible to keep the cells living for indefinite periods of time with regular growth media changes and controlling the incubation temperature and gas ratios. The cells can be monitored in real time during the incubation using the present subject matter.

During a drug screen assay of the present approach, electrophysiological parameters such as action potential, action potential periodicity and amplitude, cell impedance, and action potential velocity can be measured to assess the viability and response of cells attached to the present apparatus when exposed to drugs, biologics, toxins, and environmental stresses such as temperature, pressure or radiation exposure. The assay is effective in measuring both cellular toxicity and efficacy by measuring electrophysiology response of various therapeutics on mammalian cells, self aligned on the micro structured NIEA microwell plates.

The above discussion is meant to be illustrative of the principle and various embodiments of the present invention. Numerous variations and modifications will become apparent to those skilled in the art once the above disclosure is fully appreciated.

What is claimed is:

1. A support comprising a surface micro structured multi electrode array having a microwell plate disposed thereon, wherein one or more wells in the microwell plate lack bottoms, wherein the microstructures form a micropattern on the surface of the support and wherein the multi electrodes are integrated onto the surface of the support.

2. The support of claim 1 wherein each well has the same number of electrodes.

3. The support of claim 1 wherein the support comprises glass, silicon, printed circuit board, Kapton, polycarbonate, or polyester.

4. The support of claim 1 wherein the electrodes have an electrical resistivity of less than 100 ohms per square.

5. The support of claim 1 wherein the electrodes are gold coated copper electrodes flash plated with gold.

6. The support of claim 1 wherein the micro pattern comprises lines with dimensions that range from 10 to 60 microns in width and 5 to 50 microns in height with channels or spaces between the lines from 20 to 70 microns.

7. The support of claim 1 wherein the micro pattern comprises lines with dimensions that range from 10 to 60 microns in width and 5 to 50 microns in height with channels or spaces between the lines.

8. The support of claim 1 wherein the micro pattern comprises lines with channels or spaces between the lines from 20 to 70 microns.

9. The support of claim 1 wherein the electrodes are spaced between the microstructures.

10. The support of claim 1 wherein the electrodes are in a pattern.

11. A method of screening cells for measuring electrophysiological induced changes in mammalian cells using a microelectrode array, comprising:
providing the support of claim 1;
introducing mammalian cells onto areas of the support having the electrodes;
exposing the mammalian cells to one or more test compounds or one or more environmental stresses; and
determining whether the one or more test compounds or the one or more environmental stresses alter the electrical activity of the cells.

12. The method of claim 11 wherein the cells are cardiomyocytes, neuron progenitors, neurons, fibroblasts, endothelial cells, epithelial cells, liver c kidney cells, pancreas cells or dorsal root ganglion cells.

13. The method of claim 11 further comprising stimulating the cells with the electrodes.

14. The method of claim 13 wherein the stimulation is before the cells are exposed to one or more test compounds.

15. The method of claim 13 wherein the stimulation is after the cells are exposed to one or more test compounds.

16. The method of claim 11 wherein the electrodes record the electrical activity of the cells.

17. The method of claim 11 wherein one or more of the electrodes are coated with a protein that binds the cells.

18. The method of claim 11 wherein prior to exposure the cells are self aligned.

19. The method of claim 11 wherein the environmental stress is temperature, pressure or radiation exposure.

20. The method of claim 11 wherein action potential, action potential periodicity and amplitude, cell impedance, or action potential velocity is determined.
